(19) United States
(12) Patent Application Publication
(10) Pub. No.: US 2008/0033014 A1
(43) Pub. Date: Feb. 7, 2008

(54) PHARMACEUTICAL COMPOSITION COMPRISING, IN COMBINATION, SAREDUTANT AND A SELECTIVE SEROTONIN REUPTAKE INHIBITOR OR A SEROTONIN/NOREPINEPHRINE REUPTAKE INHIBITOR

(75) Inventors: Guy Griebel, Fontenay Aux Roses (FR); Caroline Cohen, Paris (FR); Caroline Louis, Paris (FR); Lisa Ann Arvanitis, Kennett Square, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/830,325

(22) Filed: Jul. 30, 2007

(30) Foreign Application Priority Data
Jul. 31, 2006 (FR) 0607050
Feb. 7, 2006 (FR) 0700863

(51) Int. Cl. A61K 31/4468; A61P 25/18; A61P 25/24

(52) U.S. Cl. 514/329

(57) ABSTRACT

A subject-matter of the present invention is pharmaceutical compositions comprising, in combination, at least one active ingredient chosen from (S)-(−)-N-[4-(4-acetamido-4-phenylpiperidin-1-yl)-2-(3,4-dichlorophenyl)butyl]-N-methylbenzamide and pharmaceutically acceptable salts there and at least one second active ingredient chosen from selective serotonin reuptake inhibitors, serotonin/norepinephrine reuptake inhibitors and pharmaceutically acceptable salts thereof.

This application claims the benefit of priority of French Patent Application No. FR06/07,050, filed Jul. 31, 2006 and French Patent Application No. FR07/00,863, filed Feb. 7, 2007.

A subject-matter of the present invention is pharmaceutical compositions comprising, in combination, at least one active ingredient chosen from (S)-(−)-N-[4-(4-acetamido-4-phenylpiperidin-1-yl)-2-(3,4-dichlorophenyl)butyl]-N-methylbenzamide and pharmaceutically acceptable salts there and at least one second active ingredient chosen from selective serotonin reuptake inhibitors (SSRI) and a serotonin/norepinephrine reuptake inhibitors (SNRI).

(S)-(−)-N-[4-(4-Acetamido-4-phenylpiperidin-1-yl)-2-(3,4-dichlorophenyl)butyl]-N-methylbenzamide, the international nonproprietary name of which is saredutant, of formula: 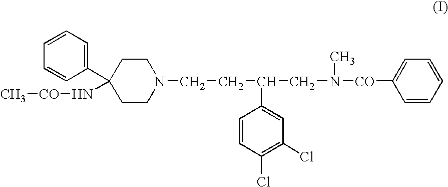
hereinafter referred to as compound A, and its pharmaceutically acceptable salts have been described as a nonpeptide antagonists of the NK₂ receptors of neurokinin A (Life Sciences, 1992, 50(15), PL101-PL106) and can be prepared according to Patents EP 0 474 5671 or U.S. Pat. No. 5,236,921.

It has now been found, surprisingly, that the combination of at least one active ingredient chosen from saredutant and pharmaceutically acceptable salts and at least one second active ingredient chosen from selective serotonin reuptake inhibitors and serotonin/norepinephrine reuptake inhibitors and pharmaceutically acceptable salts thereof may significantly enhance the pharmacological effects of each of the active compounds used alone, in particular the antidepressant effects.

In another aspect of this invention there is provided a pharmaceutical composition comprising a combination of at least one active ingredient chosen from saredutant and pharmaceutically acceptable salts and at least one second active ingredient chosen from selective serotonin reuptake inhibitors and serotonin/norepinephrine reuptake inhibitors further in combination with at least one pharmaceutically acceptable excipient.

In further aspects of this invention there are also provided methods of treatment of various mood disorders and anxiety disorders using the compositions of this invention as specifically disclosed hereinbelow.

The salts are the salts with conventional pharmaceutically acceptable inorganic or organic acids, such as the hydrochloride, the hydrobromide, the sulfate, the hydrogensulfate, the dihydrogenphosphate, the methanesulfonate, the methyl sulfate, the acetate, the oxalate, the maleate, the fumarate, the succinate, the naphthalene-2-sulfonate, the glyconate, the gluconate, the citrate, the isethionate, the benzenesulfonate or the para-toluenesulfonate.

The term “selective serotonin reuptake inhibitor” (SSRI) is understood to mean a compound such as, for example:
(±)-N-methyl-3-phenyl-3-[4-(trifluoromethyl)phenoxy]propan-1-amine, the international nonproprietary name of which is fluoxetine, of formula: 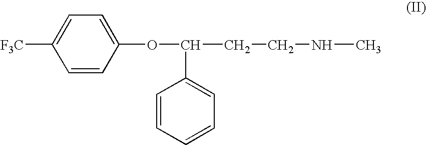
   hereafter referred to as compound B, and its pharmaceutically acceptable salts, which can be prepared according to U.S. Pat. No. 4,314,081;
1-[3-dimethylamino)propyl]-1-(4-fluorophenyl)-1,3-dihydro-2-benzofuran-5-carbonitrile, the international nonproprietary name of which is citalopram, and its pharmaceutically acceptable salts, which can be prepared according to U.S. Pat. No. 4,136,193;
(1S)-cis-4-(3,4-dichlorophenyl)-1,2,3,4-tetrahydro-N-methyl-1-naphthaleneamine, the international nonproprietary name of which is sertraline, and its pharmaceutically acceptable salts, which can be prepared according to U.S. Pat. No. 4,536,518; and
5-methoxy-1-[4-(trifluoromethyl)phenyl]-1-pentanone O-(2-aminoethyl)oxime, the international nonproprietary name of which is fluvoxamine, and its pharmaceutically acceptable salts, which can be prepared according to U.S. Pat. No. 4,085,225.

The term “serotonin/norepinephrine reuptake inhibitor” (SNRI) is understood to mean a compound such as, for example:
1-[2-dimethylamino-1-(4-methoxyphenyl)ethyl]cyclohexan-1-ol, the international nonproprietary name of which is venlafaxine, and its pharmaceutically acceptable salts, which can be prepared according to EP Patent, EP 0 112 669;
(+)-(S)-N-methyl-3-(1-naphthyloxy)-3-(thiophen-2-yl)propan-1-amine, the international nonproprietary name of which is duloxetine, and its pharmaceutically acceptable salts, which can be prepared according to EP Patent, EP 0 273 658; and
(1R,2R)-2-(aminomethyl)-N,N-diethyl-1-phenylcyclopropane-1-carboxamide, the international nonproprietary name of which is milnacipran, and its pharmaceutically acceptable salts, which can be prepared according to U.S. Pat. No. 4,478,836.

It has now been found, surprisingly, that the combination of at least one active ingredient chosen from saredutant and pharmaceutically acceptable salts there of and at least one second active ingredient chosen from selective serotonin reuptake inhibitors and serotonin/norepinephrine reuptake inhibitors may significantly enhance the pharmacological effects of each of the active compounds used alone, in particular the antidepressant effects.

Thus, the pharmaceutical compositions comprising such combinations can be of use in the manufacture of medicaments intended for the prevention and treatment of mood disorders, such as major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, such as panic attack, agoraphobia, social phobia, obsessive-compulsive disorder, post-traumatic stress condition, acute stress condition, generalized anxiety disorder or anxiety disorder induced by a substance.

In particular, the pharmaceutical compositions comprising such combinations can be of use in the manufacture of medicaments intended for the prevention and treatment of a major depressive disorder.

In particular again, the pharmaceutical compositions comprising such combinations can be of use in the manufacture of medicaments intended for the treatment of sexual dysfunctions associated with a major depressive disorder.

the term “sexual dysfunctions” is understood to mean any pathology as defined by the American Psychiatric Association—DSM-IV-TR. Diagnostic and Statistical Manual of Mental Disorders, 4th edition, revised text (Washington D.C., 2000), pages 617-654, and which includes disorders of sexual desire (that is to say, the disorder: fall in sexual desire, and the disorder: sexual aversion), disorders of sexual arousal (that is to say, female sexual arousal disorder and male erectile disorder), orgasmic disorders (that is to say, female orgasmic disorder, male orgasmic disorder and premature ejaculation), painful sexual disorders (that is say, dyspareunia and vaginismus), sexual dysfunction due to a general medial condition, sexual dysfunction induced by a substance and sexual dysfunctions not otherwise specified.

Thus, according to one of its aspects, a subject-matter of the present invention is pharmaceutical compositions comprising, in combination, at least one active ingredient chosen from saredutant and pharmaceutically acceptable salts and at least one second active ingredient chosen from selective serotonin reuptake inhibitors and serotonin/norepinephrine reuptake inhibitors, and optionally also at least one pharmaceutically acceptable excipient.

In particular, a subject-matter of the present invention is pharmaceutical compositions comprising, in combination, at least one active ingredient chosen from saredutant and pharmaceutically acceptable salts and at least one second active ingredients chosen from selective serotonin reuptake inhibitors and at least one pharmaceutically acceptable excipient.

In particular again, a subject-matter of the present invention is pharmaceutical compositions comprising, in combination, at least one active ingredient chosen from saredutant and pharmaceutically acceptable salts and at least one second active ingredient chosen from selective serotonin reuptake inhibitors chosen from fluoxetine, citalopram, sertraline and fluvoxamine and pharmaceutically acceptable salts thereof and also at least one pharmaceutically acceptable excipient.

In particular again, a subject-matter of the present invention is pharmaceutical compositions comprising, in combination, at least one active ingredient chosen from saredutant and pharmaceutically acceptable salts and at least one second active ingredient chosen from fluoxetine and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from citalopram and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from sertraline and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from fluvoxamine and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from serotonin/norepinephrine reuptake inhibitors, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from serotonin/norepinephrine reuptake inhibitors chosen from venlafaxine, duloxetine and milnacipran and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from venlafaxine and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from duloxetine and pharmaceutically acceptable salts thereof, and also at least one pharmaceutically acceptable excipient.

In particular again, a subject-matter of the present invention is pharmaceutical compositions comprising, in combination, at least one active ingredient chosen from saredutant and pharmaceutically acceptable salts thereof and at least one second active ingredient chosen from milnacipran and pharmaceutically acceptable salts thereof, and also at least one pharmaceutically acceptable excipient.

According to another of its aspects, a subject-matter of the present invention is the combination of at least one active ingredient chosen from saredutant and pharmaceutically acceptable salts thereof and at least one second active ingredient chosen from selective serotonin reuptake inhibitors and serotonin/norepinephrine reuptake inhibitors.

In particular, a subject-matter of the present invention is the combination of at least one active ingredient chosen from saredutant and pharmaceutically acceptable salts thereof and at least one second active ingredient chosen from selective serotonin reuptake inhibitor chosen from fluoxetine, citalopram, sertraline and fluvoxamine and pharmaceutically acceptable salts thereof.

In particular again, a subject-matter of the present invention is the combination of at least one active ingredient chosen from saredutant and pharmaceutically acceptable salts thereof and at least one second active ingredient chosen from serotonin/norepinephrine reuptake inhibitor chosen from venlafaxine, duloxetine and milnacipran and pharmaceutically acceptable salts thereof.

According to another of its aspects, a subject-matter of the present invention is the use of a pharmaceutical composition comprising, in combination, at least one active ingredient chosen from saredutant and pharmaceutically acceptable salts thereof and at least one second active ingredient chosen from selective serotonin reuptake inhibitors and serotonin/norepinephrine reuptake inhibitors, in the manufacture of medicaments intended for the prevention and treatment of mood disorders chosen from major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, such as panic attack, agoraphobia, social phobia, obsessive-compulsive disorder, post-traumatic stress condition, acute stress condition, generalized anxiety disorder or anxiety disorder induced by a substance.

In particular, a subject-matter of the present invention is the use of a pharmaceutical composition comprising, in combination, at least one active ingredient chosen from saredutant and pharmaceutically acceptable salts thereof and at least one second active ingredient chosen from selective serotonin reuptake inhibitors and serotonin/norepinephrine reuptake inhibitors, in the manufacture of medicaments intended for the prevention and the treatment of a major depressive disorder.

In particular again, a subject-matter of the present invention is the use of a pharmaceutical composition comprising, in combination, saredutant or one of its pharmaceutically acceptable salts with a selective serotonin reuptake inhibitor or with a serotonin/norepinephrine reuptake inhibitor, in the manufacture of medicaments intended for the treatment of sexual dysfunctions associated with a major depressive disorder.

According to another of its aspects, a subject-matter of the present invention is the use of the combination of at least one active ingredient chosen from saredutant and pharmaceutically acceptable salts thereof and at least one second active ingredient chosen from selective serotonin reuptake inhibitors and serotonin/norepinephrine reuptake inhibitors and pharmaceutically acceptable salts thereof, in the manufacture of medicaments intended for the prevention and treatment of mood disorders chosen from major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, such as panic attack, agoraphobia, social phobia, obsessive-compulsive disorder, post-traumatic stress condition, acute stress condition, generalized anxiety disorder or anxiety disorder induced by a substance.

The excipients are chosen, according to the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principles can be administered in unit administration form, as a mixture with conventional pharmaceutical excipients, to animals and human beings for the prevention or treatment of the above disorders or diseases.

The appropriate unit administration form comprise oral forms, such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms and implants. For topical application, the compounds according to the invention can be used in creams, gels, ointments or lotions.

In the pharmaceutical compositions of the present invention, the active principle or the active principles are generally formulated in dosage units containing from 2.5 to 500 mg, advantageously from 10 to 250 mg and preferably from 10 to 150 mg of the said active principle per dosage unit for daily administrations. There may be particular case in which higher or lower dosages are appropriate, such dosages to not depart from the context of the invention. According to the usual practice, the dosage that is appropriate for each patient is determined by the doctor according to the mode of administration and the weight and response of the said patient.

According to another aspect of the invention, the compound A and the other active principle according to the invention can be administered simultaneously, separately or spread out over time.

The term “simultaneous” is understood to mean the administration of the compounds of the composition according to the invention comprise within one and the same pharmaceutical form.

The term “separate” is understood to mean the administration, at the same time, of the two compounds of the composition according to the invention, each comprised within a separate pharmaceutical form.

The term “spread out over time” is understood to mean the successive administration of the first compound of the composition according to the invention, comprised within a pharmaceutical form, and then of the second compound of the composition according to the invention, comprised within a separate pharmaceutical form.

In the case of this “spread out over time”, the period of time elapsed between the administration of the first compound of the composition according to the invention and the administration of the second compound of the same composition according to the invention generally does not exceed 24 hours.

The unit pharmaceutical forms comprising either just one of the constituent compounds of the composition according to the invention or the combination of the 2 compounds which can be employed in the various types of use described above may, for example, be appropriate for oral, nasal, parenteral or transdermal administration.

Consequently, in the case of a “separate” use and of a use “spread out over time”, two separate pharmaceutical forms may be intended for the same route of administration or for a different route of administration (oral and transdermal or oral and nasal or parenteral and transdermal, and the like).

The invention thus also relates to a kit comprising the compound A and the at least one second active ingredient according to the invention in which the said compound A and the at least one second active ingredient according to the invention are in separate compartments and in similar or different packagings and are intended to be administered simultaneously, separately or spread out over time.

Specifically and without implied limitation, the enhancement in the pharmacological effects of a combination according to the invention of the compound A and of fluoxetine (compound B) have been demonstrated in animals.

### EXAMPLE 1

The in-vivo rat test DRL-72 s (Differential Reinforcement of Low-rate-72 seconds) is used according to the technique described by C. louis et al., Neuropsychopharmacology, 2006, 1-8.

Effects compared with regard to the percentage of rewards obtained (reinforced presses) with respect to the total number of presses by the rate after intraperitoneal administration of the compound A alone, of the compound B alone and of the compound A+compound B combination versus the control (solvent alone).

Beforehand, the minimum active doses of the compound A alone and of the compound B alone in the DRL-72 s test were determined, namely:
compound A: 10 mg/kg intraperitoneally;
compound B: 5 mg/kg intraperitoneally.

For the present study, a weakly active dose of compound A alone and an inactive dose of compound B alone and of compound A+compound B were selected.

The compound A alone at the dose of 3 mg/kg and the compound B alone at the dose of 2.5 mg/kg were dissolved in a 0.9% (weight/volume) aqueous sodium chloride solution comprising 0.1% (v/v) Tween 80® and administered intraperitoneally at a final volume of 1 ml/kg.

The combination was administered intraperitoneally by two simultaneous administrations of the compound A (3 mg/kg) and then of the compound B (2.5 mg/kg).

The doses of the compounds are expressed in the free base form.

For the requirements of the test, the effect of the compound A alone, the effect of the compound B alone and the effect of the compound A+compound B combination, compared with the effect of the solvent (control), are measured for each animal.

Each rat (n=8) thus receives four injections spread out over time, namely the solvent (control), the compound A alone, the compound B alone and the compound A+compound B combination.

The results obtained are collated in Table I and are expressed as a percentage of rewards obtained with respect to the total number of presses over the duration of the test (1 hour), in the mean±SEM (standard error of the mean) form.

TABLE I

| | % reinforced presses/total number of |
|---|---|
| | presses (n = 8 rats) |
| Solvent control | 3.07 ± 0.48% |
| Compound A, 3 mg/kg | 6.41 ± 1.73% |
| Compound B, 2.5 mg/kg | 3.82 ± 0.76% |
| Compound, A 3 mg/kg + | 11.01 ± 2.6%* |
| Compound B, 2.5 mg/kg | |

*p < 0.05 versus control

The results obtained show that:
the compound A, administered alone at the dose of 3 mg/kg, only slightly modifies the percentage number of rewards obtained with respect to the control; furthermore, this increase is not statistically significant;
the compound B, administered alone at the dose of 2.5 mg/kg, does not modify the percentage number of rewards obtained with respect to the control;
the combination of the compound A and the compound B markedly increases the percentage number of rewards obtained with respect to the control and this increase is statistically significant.

Thus, the combination of the compound A and the compound B according to the invention unexpectedly shows its positive effects on the behavior of the animals in this test, making it possible to confirm the antidepressant potential of the combination for a therapeutic application.

### EXAMPLE 2

A pharmaceutical composition in accordance with this invention in the form of a capsule comprising 30 mg of saredutant was prepared including the following pharmaceutically acceptable excipients.

| Saredutant (expressed as base) | 30.0 | mg |
|---|---|---|
| Lactose monohydrate (200 mesh) | QSP 400.0 | mg |
| Croscarmellose sodium | 8.0 | mg |
| Magnesium stearate | 4.0 | mg |
| Purified wafer* | QS | |
| Size-0 opaque hard capsule, filled with | 400.0 | mg |

*drying evaporated after moist grainy effect.

### EXAMPLE 3

A pharmaceutical composition in accordance with this invention in the form of a capsule comprising 100 mg of saredutant was prepared including the following pharmaceutically acceptable excipients.

| Saredutant (expressed as base) | 100.0 | mg |
|---|---|---|
| Lactose monohydrate (200 mesh) | QSP 400.0 | mg |
| Croscarmellose sodium | 8.0 | mg |
| Magnesium stearate | 4.0 | mg |
| Purified water* | QS | |
| Size-0 opaque hard capsule, filled with | 400.0 | mg |

*drying evaporated after moist grainy effect.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A pharmaceutical composition comprising, in combination, at least one active ingredient chosen from saredutant and pharmaceutically acceptable salts thereof and at least one second active ingredient chosen from selective serotonin reuptake inhibitors, serotonin/norepinephrine reuptake inhibitors, and pharmaceutically acceptable salts thereof, further in combination with at least one pharmaceutically acceptable excipient.

2. The composition according to claim 1, wherein the at least one active ingredient is chosen from saredutant and pharmaceutically acceptable salts thereof and the at least one second active ingredient is chosen from selective serotonin reuptake inhibitors and pharmaceutically acceptable salts thereof.

3. The composition according to claim 2, wherein the selective serotonin reuptake inhibitors are chosen from fluoxetine, citalopram, sertraline, fluvoxamine and pharmaceutically acceptable salts thereof.

4. The composition according to claim 2, wherein the selective serotonin reuptake inhibitors are chosen from fluoxetine and pharmaceutically acceptable salts thereof.

5. The composition according to claim 2, wherein the selective serotonin reuptake inhibitors are chosen from citalopram and pharmaceutically acceptable salts thereof.

6. The composition according to claim 2, wherein the selective serotonin reuptake inhibitors are chosen from sertraline and pharmaceutically acceptable salts thereof.

7. The composition according to claim 2, wherein the selective serotonin reuptake inhibitors are chosen from fluvoxamine and pharmaceutically acceptable salts thereof.

8. The composition according to claim 1, wherein the at least one active ingredient is chosen from saredutant and pharmaceutically acceptable salts thereof and the at least one second active ingredient is chosen from serotonin/norepinephrine reuptake inhibitors and pharmaceutically acceptable salts thereof.

9. The composition according to claim 8, wherein the serotonin/norepinephrine reuptake inhibitors are chosen from venlafaxine, duloxetine, milnacipran and pharmaceutically acceptable salts thereof.

10. The composition according to claim 8, wherein the serotonin/norepinephrine reuptake inhibitors are chosen from venlafaxine and pharmaceutically acceptable salts thereof.

11. The composition according to claim 8, wherein the serotonin/norepinephrine reuptake inhibitors are chosen from duloxetine and pharmaceutically acceptable salts thereof.

12. The composition according to claim 8, wherein the serotonin/norepinephrine reuptake inhibitors are chosen from milnacipran and pharmaceutically acceptable salts thereof.

13. A combination of at least one active ingredient chosen from saredutant and pharmaceutically acceptable salts thereof and at least one second active ingredient chosen from selective serotonin reuptake inhibitors, serotonin/norepinephrine reuptake inhibitors and pharmaceutically acceptable salts thereof.

14. The combination according to claim 13, wherein the selective serotonin reuptake inhibitors are chosen from fluoxetine, citalopram, sertraline, fluvoxamine and pharmaceutically acceptable salts thereof.

15. The combination according to claim 13, wherein the serotonin/norepinephrine reuptake inhibitors are chosen from venlafaxine, duloxetine, milnacipran and pharmaceutically acceptable salts thereof.

16. A method of treatment of a mood disorder in a patient comprising administering to the patient a therapeutically effective amount of a combination of at least one active ingredient chosen from saredutant and pharmaceutically acceptable salts thereof and at least one second active ingredient chosen from selective serotonin reuptake inhibitors, serotonin/norepinephrine reuptake inhibitors and pharmaceutically acceptable salts thereof and optionally in combination with one or more pharmaceutically acceptable excipients.

17. The method according to claim 16, wherein the at least one active ingredient is chosen from saredutant and pharmaceutically acceptable salts thereof and is administered in combination with at least one second active ingredient chosen from selective serotonin reuptake inhibitors and pharmaceutically acceptable salts thereof.

18. The method according to claim 18, wherein the selective serotonin reuptake inhibitors are chosen from fluoxetine, citalopram, sertraline, fluvoxamine and pharmaceutically acceptable salt thereof.

19. The method according to claim 16, wherein the at least one active ingredient is chosen from saredutant and pharmaceutically acceptable salts thereof and is administered in combination with at least one second active ingredient chosen from serotonin/norepinephrine reuptake inhibitors and pharmaceutically acceptable salts thereof.

20. The method according to claim 19, wherein the serotonin/norepinephrine reuptake inhibitors are chosen from venlafaxine, duloxetine, milnacipran and pharmaceutically acceptable salts thereof.

21. The method according to claim 16, wherein the mood disorder is major depressive disorder.

22. The method according to claim 21, wherein the major depressive disorder is sexual dysfunctions associated with a major depressive disorder.

23. The method according to claim 16, wherein the mood disorder is resistant depressive disorder.

24. The method according to claim 16, wherein the mood disorder is dysthymic disorder.

25. The method according to claim 16, wherein the mood disorder is depressive disorder not otherwise specified.

26. The method according to claim 16, wherein the mood disorder is bipolar I disorder.

27. The method according to claim 16, wherein the mood disorder is bipolar II disorder.

28. The method according to claim 16, wherein the mood disorder is cyclothymic disorder.

29. The method according to claim 16, wherein the mood disorder is bipolar disorder not otherwise specified.

30. The method according to claim 16, wherein the mood disorder is mood disorder due to a general medical condition.

31. The method according to claim 16, wherein the mood disorder is mood disorder induced by a substance.

32. The method according to claim 16, wherein the mood disorder is mood disorder not otherwise specified.

33. A method of treatment of an anxiety disorder in a patient comprising administering to the patient a therapeutically effective amount of a combination of at least one active ingredient chosen from saredutant and pharmaceutically acceptable salts thereof and at least one second active ingredient chosen from selective serotonin reuptake inhibitors, serotonin/norepinephrine reuptake inhibitors and pharmaceutically acceptable salts thereof optionally in combination with one or more pharmaceutically acceptable excipients.

34. The method according to claim 33, wherein the at least one active ingredient is chosen from saredutant and pharmaceutically acceptable salts thereof and is administered in combination with at least one second active ingredient chosen from selective serotonin reuptake inhibitors and pharmaceutically acceptable salts thereof.

35. The method according to claim 34, wherein the selective serotonin reuptake inhibitors are chosen from fluoxetine, citalopram, sertraline, fluvoxamine and pharmaceutically acceptable salts thereof.

36. The method according to claim 33, wherein the at least one active ingredient is chosen from saredutant and pharmaceutically acceptable salts thereof and is administered in combination with at least one second active ingredient chosen from serotonin/norepinephrine reuptake inhibitors and pharmaceutically acceptable salts thereof.

37. The method according to claim 36, wherein the serotonin/norepinephrine reuptake inhibitor is chosen from venlafaxine, duloxetine, milnacipran and pharmaceutically acceptable salts thereof.

38. The method according to claim 33, wherein the anxiety disorder is panic attack.

39. The method according to claim 33, wherein the anxiety disorder is agoraphobia.

40. The method according to claim 33, wherein the anxiety disorder is social phobia.

41. The method according to claim 33, wherein the anxiety disorder is obsessive-compulsive disorder.

42. The method according to claim 33, wherein the anxiety disorder is post-traumatic stress condition.

43. The method according to claim 33, wherein the anxiety disorder is acute stress condition.

44. The method according to claim 33, wherein the anxiety disorder is generalized anxiety disorder.

45. The method according to claim 33, wherein the anxiety disorder is anxiety disorder induced by a substance.
